(19) United States
(12) Patent Application Publication
(10) Pub. No.: US 2024/0306569 A1
(43) Pub. Date: Sep. 19, 2024

(54) A DATA COLLECTION AND MONITORING SYSTEM, A CONTROLLED ENVIRONMENT FARMING SYSTEM, DEVICES AND RELATED METHODS

(71) Applicant: Ocado Innovation Limited, Hatfield, Hertfordshire (GB)

(72) Inventors: Andrew INGRAM-TEDD, Hatfield, Hertfordshire (GB); Stephen MILLWARD, Hatfield, Hertfordshire (GB); Benjamin Arthur Portnoy NOAR, Hatfield, Hertfordshire (GB)

(73) Assignee: Ocado Innovation Limited, Hatfield, Hertfordshire (GB)

(21) Appl. No.: 18/262,217

(22) PCT Filed: Jan. 21, 2022
(86) PCT No.: PCT/EP2022/051341

§ 371 Date: Jul. 20, 2023

(30) Foreign Application Priority Data
Jan. 21, 2021 (GB) 2100791.9

(51) Int. Cl. A01G 31/04; G01N 33/00; G06F 30/20

(52) U.S. Cl. CPC A01G 31/045; G01N 33/0098; G06F 30/20

(57) ABSTRACT

A data collection and monitoring system for assessing a crop of living organisms in a controlled environment farming system is provided. The collection and monitoring system includes a data collection device having an imaging capability, a depth sensor capability, and one or more environment sensors; and a data processor receiving collected data from the data collection device, wherein based on collected data the data processor provides topographical mapped information of an imaged area combined with collected environmental data as an output.

### FIELD OF THE INVENTION

The invention relates to a farming system, method and related devices. More specifically the invention relates to data collection and monitoring within indoor or controlled environment farms, such as vertical farms.

### INTRODUCTION

Conventional systems and methods for growing crops are well known. Most require large areas of land and need to be positioned in appropriate locations for the conditions required for the crops to be grown.

Indoor farming under artificial lights is gaining popularity for a large number of crops. There are many benefits associated with indoor farming, such as reduced needs for water, fertilisers and pesticides as well as increased control of taste, texture and other features of the crop.

More recently, advanced farming techniques such as hydroponics, aeroponics and other such cultivation systems have led to the ability to grow high quality crops indoors with very high utilisation of lighting, water and fertiliser. These systems have however been less efficient in terms of land use, capital and labour.

The benefits from controlled environment, indoor and or vertical farming systems are likely to become more pronounced as improved infrastructure components such as more efficient and cheaper lights, and cheaper electricity become available.

Commercially viable indoor farming solutions require maximisation of efficient use of space and other resources such as lighting, fluids, and labour.

Presently, mainly short plants, such as herbs and leafy greens are grown indoors under artificial light.

An example of an indoor farm is disclosed in WO202030825 A1 “Hydroponics Growing System and Method” (Ocado Innovations Ltd.), the contents of which are incorporated herein by reference. Seeds are pre-treated and germinated in a ‘high care’ portion to reduce contamination during germination. Seedlings are then moved to a growing room in support vehicles containing growing which trays move along a frame or rack as the crop grows and until the crop is ready for harvesting. The system disclosed includes illumination apparatus above each growing station, and a recirculating irrigation system for providing nutrients to a growing crop. The irrigation system uses mains water blended with nutrients, which is pumped to the growing crop. Water which drains from the racks is reintroduced to the water blend to minimise waste water.

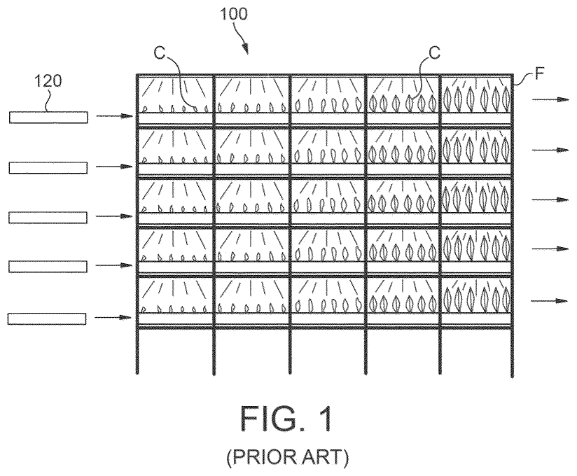

WO'825 describes an apparatus 100 for use in a hydroponic growing system, as shown in FIG. 1. The apparatus 100 is located within a high care facility within the hydroponic growing system. The apparatus comprises a frame F of vertical members and horizontal members supporting horizontal tracks or guideways on which a set of growing vehicles 120 are mounted. The growing vehicles each contain a number of growing trays in which plants or crops C are accommodated whilst they grow.

In WO'825, the size of the facility is limited by the size of the building and frame within. The temperature, humidity and wind speed are controlled within the high care facility. The environment is substantially the same for each growing tray. Accordingly, typically the same crop or type of crop is grown in each track. In the arrangement described in WO'825 it is only possible to access trays sequentially from each track or guideway. Mechanical failure of a track may render a large portion of the facility inaccessible until it is repaired.

Some commercial and industrial activities require systems that enable the storage and retrieval of a large number of different products.

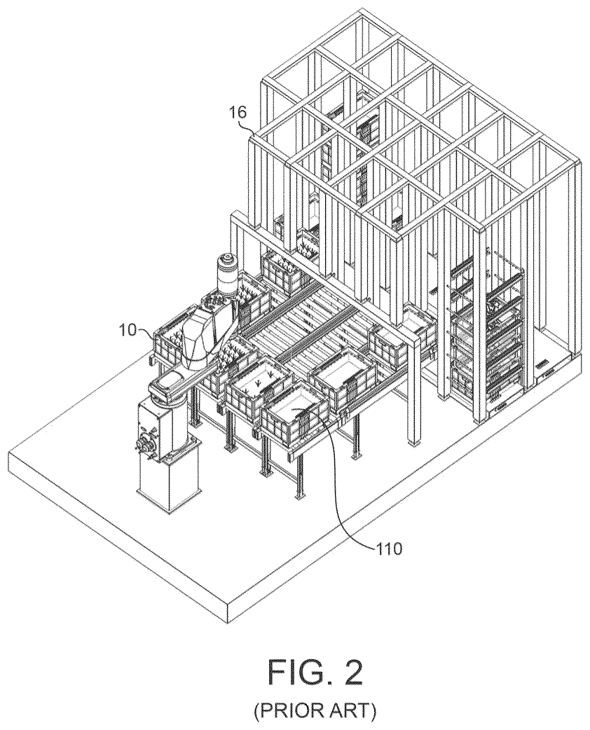

Another example of an indoor farm is disclosed in WO2018050816A1 “Growing Systems And Methods” (Ocado Innovations Ltd.), the contents of which are incorporated herein by reference. As shown in FIG. 2, WO'816 describes a growing system where plants are grown in containers 110, and the containers are stored in stacks. Above the stacks, load handling devices run on a grid network of tracks 16 and take containers from the stacks and deposit them at alternative locations in the stacks or at work stations 10. The containers are provided with services and deployable lighting means. The provision of such lighting means within individual containers rather than across the system as a whole, allows for flexibility in storage whilst reducing cost and inefficiency and enables multiple types of living organisms to be grown in a single area. As the growing conditions are controllable for each container, the facility can accommodate a number of different crops and living organism. Any container may be accessed and processed at a work station, however, it may be inefficient to access some containers, for example, those located at the bottom of a stack.

The present invention aims to further develop indoor growing, vertical or controlled environment farming systems and methods. In particular, the present invention aims to further the art in data driven crop monitoring, and use of data to improve productivity of controlled environment farms. It follows that improvements in quality, freshness, maturity, size, consistency etc. as characteristics of the crop may be achieved as a result.

It will be appreciated that the present invention also aims to maximise the yield, improve efficiency in terms of use of assets, resources and services required by the crop. For example, but not limited to efficiencies, may comprise: reduced needs for water; reduced needs for fertilisers and pesticides; increase in the control of taste; increase in the control texture and other features of the crop; efficiencies in the use of artificial lighting; efficiencies in maintenance of the facilities; improved utilisation of space, improved safety, an increase in automation and corresponding decrease in labour. Overall, the present invention aims to address issues enabling more growth in a smaller space with less electricity consumption, less capital expenditure, less maintenance and less labour cost.

Furthermore, the benefits from controlled environment or vertical farming systems are likely to become more pronounced as improved infrastructure components such as more efficient and cheaper lights, and cheaper electricity become available.

Live green plants absorb radiation in the photosynthetically active radiation (PAR) spectral region to use as an energy source to the process of photosynthesis to grow. Leaf cells have also evolved to re-emit solar radiation in the near-infrared (NIR) spectral region because the photon energy at wavelengths longer than about 700 nanometres is too small to synthesize organic molecules. A strong absorption at these wavelengths would only result in overheating the plant and possibly damaging the tissues. The cell structure of the leaves, on the other hand, strongly reflects near-infrared light (700-1100 nanometres). Accordingly, live green plants appear relatively dark in the PAR spectral region and relatively bright in the near-infrared. The more leaves a plant has, the more these wavelengths of light are affected, respectively.

The normalized difference vegetation index (NDVI) is an indicator that can be used to analyse multispectral image measurements and assessing whether or not the image contains live green vegetation. Typically, NDVI takes place via ‘big data’ image processing systems, often using images from a space platform. These systems may use pixel- or object-based algorithms to assess vegetation, health, evapotranspiration, and other ecosystem functions. More recently drones are used to capture images which are analysed using NDVI to compare data and recognize crop health issues. Also, it has been shown that modified (red green blue) RGB cameras may obtain results similar to those obtained from the multispectral cameras.

NDVI is directly related to the photosynthetic capacity of the plant canopy. Higher values of NDVI indicate more dense or healthier foliage. NDVI may be determined by comparing the reflectance of red (visible) light with near-infrared (NIR) light:

$$NDVI = \frac{( NIR - Red )}{( NIR + Red )}$$

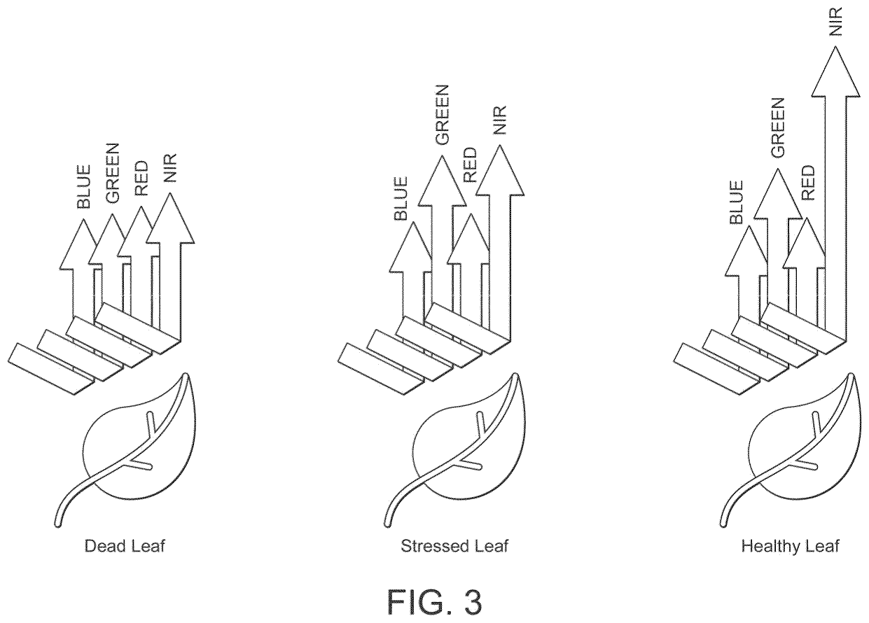

Diagrammatically, FIG. 3 compares the reflectance of light in visible blue, green, visible red and near-infrared spectral regions and shows examples of typical dead, stressed and healthy leaves. As shown, for a dead leaf, the level of reflectance is approximately equal as no spectral energy is being absorbed by the leaf. For the live leaves i.e. stressed and healthy, approximately equal amounts of blue light and red light are absorbed, while green and NIR are reflected, and for the stressed leaf the green and NIR reflectance are approximately equal, while for the healthy leaf the NIR is strongly reflected.

The present disclosure describes devices, systems, and methods for improving data collection and analysis.

### SUMMARY OF THE INVENTION

Aspects of the invention are set out in the accompanying claims.

### Data Collection and Analysis System

A data collection and monitoring system for assessing a crop of living organisms in a controlled environment farming system is provided. The collection and monitoring system comprising: a data collection device having an imaging means, and one or more environment sensors; and a data processor means receiving collected data from the data collection device, wherein based on collected data the data processor means provides mapped information of an imaged area combined with collected environmental data as an output.

The data collection device may collect or capture images of a crop. These images may be used by the data processor to provide mapped information about the crop. The data collection device may collect or survey the crop with a depth sensor means. In this way, the distance of the crop below the collection device may be known. From knowing the level of a growing tray supporting the crop, the height of the crop may be calculated. The image data and the depth or height data may be combined by the processor to provide topographical mapped information. The data collection device also collects environmental data. In this way, the environmental data may be known for the time when image data and height data are collected. It will be appreciated that each of the types of data (image, depth and environmental) are collected together to be used as inputs to the data processor to provide an output.

The data analysis facility or processor may process and use the collected data to provide information about the crop based on identified characteristics compared with the environmental conditions. Conveniently the information is mapped. In this way, a user may review the information and more readily understand how variations in conditions over an area result in variations in the crop, or identify problems with the crop that may require actions to be taken within the farm to address any issues or to produce a crop of expected quantity and quality according to a particular demand. Or a processor may use the mapped array for further processing.

In this way, the crop produced may be directly correlated to the conditions under which the crop is grown. It will be appreciated that data is collected non-invasively, i.e. without causing any interference or disturbance to the crop.

The imaging means may comprise a polarising means, a a wide angle lens, a fish-eye lens, or a LIDAR device for capturing data in three-dimensions.

The imaging means may capture wide spectrum images comprising, visible light and or non-visible light.

The non-visible light may be ultra-violet (UV), IR (infra-red), near-IR (infra-red) or thermal infra-red.

It will be appreciated that lighting in a vertical farm for growing a crop of leafy plants may be tuned to provide the spectrum of light which is used by the plants to grow. In such a vertical farm, imaging means may similarly be selected to collect or capture images of particular spectrums which are most useful for identifying the monitoring the crop. The imaging means may collect data across a wide range of light frequencies representing typical reflectance ranges of the crop. The polarising means may be a polarising filter, or a polarising sensor e.g. Polarsens TM, having a polariser array allowing multiple degrees and directions of polarisation to be detected at one time. In some circumstances, light polarisation may assist with edge-based object recognition during processing of images, particularly for example, in distinguishing individual leaves within a canopy. In such circumstances isolating reflected light by light wave orientation may assist in identifying the plane of individual leaves.

The one or more environmental sensors may comprise: wind sensor means; CO₂ sensor means; temperature sensor means; humidity sensor means; pH sensor means; conductivity sensor means; a combined CO₂/Temperature/Humidity sensor; a combined temp/pressure/humidity sensor; and or pressure sensor means.

It will be appreciated that the data collection device may comprise a number of different sensors, and the number and type of sensors are not limited to those described here. Some sensors may detect a combination of various conditions within the environment. The sensors used to collect environmental conditions may correspond to the conditions which are controllable within the vertical farm. The sensors may comprise additional types which detect conditions which are not controllable within the vertical farm but impact on the crop.

The data collection device may further comprise a probe having one or more of imaging means, a depth sensor means, one or more environmental sensors, and or a location sensor means located towards the distal end of the probe. The probe may be retractable. The retractable probe may be a telescoping probe.

Thus, it may be possible to collect data from amongst the crop. Or it may be possible to reach areas of the crop which are otherwise obscured by the canopy, taller adjacent plants, or other parts of the crop. Alternatively, it may be possible to collect more detailed information and data about a particular area of the crop, for example, to assess a particular issue that is identified from a wider view.

It will be appreciated that the data may be collected for a wide area, or for more focused regions of the crop within the vertical farm.

The data collection device may further comprise movement means for moving within an area or volume of operation.

The data collection device may be able to move within the vertical farm. The data collection device may collect data from different positions within the farm. For some areas, the information may be collected from different directions or angles, thereby providing a more detailed or complete map of the area. This approach may also provide more confidence in the data as correlations should be seen.

The data collection device may be moved to cover substantially the entire growing area of the vertical farm.

The movement means may comprise motor and wheels for moving along a track or rail system. Alternatively the data collection device may be mounted on a drone.

In other arrangements, the location of the collection device may be fixed. When fixed, the sensors may be in a distributed arrangement to collect data over a wide area of the vertical farm.

The data collection device may further comprise a depth sensor means, and the data processor may provide topographical mapped information of an imaged area combined with collected environmental data as an output.

The depth sensor means may comprise an acoustic sensor. The imaging means and depth sensor are combined to capture data in three-dimensions. The acoustic sensor may be an ultrasound sensor.

By combining the image data and the depth data it is possible to provide a three-dimensional or topographical information set. By capturing data in three-dimensions, it is possible to build up as complete an examination of the crop as possible. A data set in three-dimensions may be capturing by using a three-dimensional imaging means, or the three-dimensional data set may be created by combining data from a more traditional two-dimensional imaging means and depth sensor means such as an ultrasonic sensor.

The data collection device may further comprise a location means.

When the data collection device is movable, it may be necessary to accurately know the location of the device. The data collection device may be used throughout a wide area or volume within the operational or production space of an indoor farming system. Accordingly, a location sensor means may be used to locate the data collection device in order to create a map of the crop within the operational area. The location sensor means may locate the device relative to other components of the indoor farming system, where the other components are fixed within the space. In this way, data may be repeatedly collected at the same location in order to build up a picture of the crop and environment at that location over time. Various means may be used to establish the location of the data collection device.

The location means may be a RFID tag. The RFID tag may interact with other fixed location readers located within an area of operation. Alternatively the location means may comprise a GPS system, using lasers to determine distance from fixed points such as walls, or a camera system within the indoor farming system but external or distal from the data collection device, a beacon system, a triangulation system, a sonar system, an infra-red system, or an ultrasonic system.

It will be appreciated that the data collection device may further comprise: a battery; a CPU; a local data storage means; a transmitter means; a receiver means; transceiver means; and or calibration means, and any other necessary hardware in order to operate within a vertical farm under control of a controller.

It will be appreciated that the data collection device may be equipped with the means necessarily to collate, store and transfer data. It will be appreciated that the device may be enabled to move within the indoor farming system in order to survey the entire, or substantially all of, the crop.

### Controlled Environment Farming System

A controlled environment farming system is provided, comprising: a data collection device, and a data processor means for use in a data collection and monitoring system, wherein the data collection device is positioned to survey an area for receiving a crop and the data processor provides an output comprising topographical mapped information and environmental data.

An indoor or controlled environment farming system is provided, where it is possible to collect data about the crop and of the environment in order to analysis and monitor the crop.

The data collection and monitoring system may be a part of a controlled environment farming system. The data collection device may be located within the growing room of a vertical farm. The specific locations or positions may be the same location each time the device collects data, or the specific locations may be different each time the device collects data. The locations may be known relative to a fixed or absolute origin within the farming system. Locations may be known relative to other objects within the farming system, for example, by location indicators such as RFID tags located on racking, or indicators located on growing trays.

The data collection device may collect data within the growing room of the vertical farm. The collected data may be time stamped and processed by the data processor to provide information about the crop. By collecting data over time, the crop may be monitored by the data collection and monitoring system.

The processor may be remotely located relative to the data collection device.

It will be appreciated that while is it contemplated that the data collection device and the data processor could be co-located, more typically, the data processor may be remotely located relative to the data collection device. In this way, the data collection device may be smaller. Further the data processor may be more easily updated, or make use of other resources. It will appreciated that remote monitoring may be provided. Some processing may occur at the point of collection which may reduce the amount of data that needs to be uploaded to other devices. Cloud processing resources or distributed networks may be used to enable access to collected data at multiple locations. Cloud processing resources may be used to manage processing demand. Collected data may be uploaded on a daily basis, therefore, there may be a peak in demand on processing resource shortly after the data is uploaded.

It is anticipated that similar collection and monitoring systems may exist in a number of geographically distributed vertical farms. It will be appreciated that separate vertical farms may share data and learnings.

The processor may be operationally in communication with the data collection device and a controller is arranged to respond to the data processor output.

In this way, the output from the data processor may feed into the control of the farm. In addition, the farm controller may receive inputs of or related to the specific farm itself, for example, size of farm, growing area, water pH used within the farm, water EC (electrical conductivity), typical volumes of fluid used, typical yield or harvest from the farm etc. Further the farm controller may receive inputs about the crop which is being monitored, such as the plant species, life-cycle details and typical growing requirements etc. Still further the controller may receive inputs of demand for the crop. Thus, a feedback loop may be created based on inputs into the farm, outputs from the farm and consequences of changes with the farm based on the data collection and monitoring system. Thus, conditions may be optimised to efficiently produce high quality crops as required.

The desired outcome of the living organisms may be provided as an initial input to the controller, and remain the same throughout the life-cycle of the crop. Or the desired outcome may change over time, for example, as a result of a change in consumer demand. Desired outcomes may comprise a crop yield, a crop delivery time post-harvest, or particular crop characteristics etc.

The characteristics of primary interest may vary depending on the type of crop. Crops may have different characteristics depending on the stage of their life-cycle and these may be used to assess the level of maturity and or readiness for harvest. Further, crops may behave differently when exposed to similar environmental conditions. Accordingly, an analysis of environmental conditions compared with crop outcomes may be made by the processor, and used by the controller of the farm.

The controller may control operation of the controlled environment farming system, operations may comprise: control of the environmental conditions for growing experiments, following growing formulas, following growing recipes; controlling a seeding schedule; controlling hatching; controlling cloning; controlling reproducing, incubating or breeding; determining health state of the crop; control of continued monitoring of the crop and progressing the crop for processing at a work station; controlling harvesting and or controlling production operations such as cultivation processes; controlling the growing environment according to operational need or demand; or controlling the growing environment according to desired crop characteristics.

The controller may change or update the environment in response to the analysis or processor output in real-time. It will be appreciated that updates and changes may occur automatically, dynamically and or in real-time.

It will be appreciated that some operations may be performed remotely from the production area or growing room, and growing trays may be transferred between rooms and environments within the farm to carry out processes.

The data collection device may self-calibrate one or more of the: imaging means, depth sensor means, environmental sensor(s) and or location means against one or more known references located within the vertical farm.

In order to maintain the integrity of the data collected, the data collection device may be calibrated against data collected at a fixed point relative to a known source of information.

It will be appreciated that it may be important to set or calibrate the imaging means for camera white balance. For example, in sunlight, photos taken with the imaging means may be quite orange, with leaves being the most orange objects. Typically, in vertical farms, the lights or LEDs emit less infrared light so images will be different to those taken in sunlight. A calibration routine “WhiteBalanceTesting” may take photos with combinations of different white balance values and adjust these when comparing with a reference image. For example, the white balance of the imaging means may be calibrated against a known image or feature on the upper edge surface of a growth tray. White balance gains being may be red=1.2 blue=2.0.

Similarly, routines and checks may be performed for the depth sensor means and the environmental sensors. The depth sensor means may be calibrated by measuring the depth of the floor or an empty growth tray at a specific location. Or, for example, a temperature sensor on the data collection device may be calibrated against one or more highly sensitive and stable temperature sensors arranged within the indoor farming system. Similarly, a wind sensor, pressure sensor, CO₂ sensor, and or humidity sensor may be calibrated.

The data collection device may interface with ID tags located within the vertical farm to determine the location of the data collection device.

The framework, racks and or trays may comprise identity tags to sense location within an area of operation.

As noted above, the data collection device may comprise location means. To compliment this arrangement, ID tags located within the vertical farm may be used to verify the position of the data collection device. Further, ID tags may be used to track position of trays of crop through the vertical farm. Each ID tag may comprise a unique identifier. For example, trays may comprise an ID tag which records the tray number and position of the tray. Accordingly, even if a crop tray is moved, the crop may still be monitored and tracked by the data collection and monitoring system. The data collection device and processor may then record CO₂, temperature, % humidity, wind speed, distance/depth and pressure in a position within the vertical farm relative to the tray. It will be appreciated, measurements may be recorded relative to other objects within the vertical farm.

The data collection device may be instructed to move to a specific tray location or RFID tag location.

The farming system may further comprise a rail system extended in at least a first direction wherein the data collection device is movably mounted on the rail system for surveying an area.

The rail system may extend above a rack of growth trays containers or bins containing a crop, thereby providing means for supporting the data collection device on a suitable vantage point for collecting data, and providing means for guiding the data collection device to positions above each tray, or other predetermined monitoring positions within the farm. It will be appreciated that the data collection device may comprise wheels or other means for moving along the rail system, or the data collection device may slide along the rail system. A rail system may be mounted on a rack or frame.

It will be appreciated that the rail system may extend in a first x-direction. Further the rail system may extend in two-dimensions, x- and y-directions. Further the rail system may extend in three-dimensions, x-y- and x-directions, to extend between levels within the rack or along each level of the rack. In this way, the data collection device may be able to reach substantially all the production areas of the indoor farm. The rail system may be above the growth trays.

It will be appreciated that one or more data collection devices may be operable within the production area of the indoor farm. For example one or more on each level.

The data collection device or sensors are fixedly mounted within the vertical farm.

As noted above the data collection device(s) may be fixed in a single location, typically fixed to the racking for supporting growing trays. Of course, in this arrangement the location of the data collection will be known. However, location devices may still be used to identify which growth trays are within proximity of the data collection device for example.

The data collection device may be arranged to direct imaging and other sensing means in particular directions over the crop. For example, the sensing means may comprise means for carrying out photo-spectroscopy type methods for measuring temp and CO₂ concentration and or relative humidity.

The farming system may further comprise one or more controllable mirrors mounted within the vertical farm wherein the controllable mirrors are pivotable to direct line-of-sight of the data collection device a larger area of the controlled environment farming system.

The mounted mirrors may be directionally controllable by a farm controller.

Using mirrors around the production area of the indoor farm, it may be possible to survey a wider area of production from a single data collection device location by reflecting and directing data signals from the area of interest to the data collection device. When the mirrors are controllable, it may be possible to direct sensor collection over an even wider area of production compared with fixed direction mirrors.

The data collection device may be mounted on a drone.

In some arrangements, data collection devices may be free to move within the indoor farm in three-dimensions. Large areas of the indoor farm may be reachable by the data collection device without the need for additional structures, such as rails etc. Accordingly, there would be correspondingly fewer surfaces to keep clean, and lower capital expense.

### Method

A method of collecting and monitoring data according is provided. The method comprising the steps of: instructing the data collection device to collect image data, depth survey data and or environment data; transmitting the collected data to the data processor, wherein the data processor uses the image data to provide a map of the imaged area, and wherein the data processor stores the map with the collected environmental data.

It will be appreciated that when depth survey data is collected then a topographical map may be provided.

The image capture, depth survey and or the environmental data collection may be collected at the same time. The location of data collection device may be recorded when data is collected and stored with the map and the collected environmental data.

In use, a data collection device is positioned, typically within a farming system. The data collection device may be instructed to collect data. The collected data is passed to the data processor to produce a topographical map of the area. The information is stored for further analysis and or later use. It will be appreciated that each type of collected data may comprise a time/date stamp. In this way, the data analysis facility may monitor the progress of a specific crop, and compare the current crop with other earlier grown crops of similar type, in order to optimise efficiency of the indoor farm and to optimise crop yield.

Collected data i.e. captured images, depth surveys and collected environment data, may be collected a plurality of times. In this way, record data may comprise average readings from the sensors (imaging means, depth sensor means and environment sensors).

It will be appreciated, that collecting the different types of data at the same time, allows a comparison actual growth of the crop with the conditions under which the crop has been grown.

Outputs of the data processor may be transmitted to a controller.

The data processor may receive inputs comprising crop data; or wherein the data processor may receive inputs comprising vertical farm data.

The processed data may be passed to the vertical farm controller.

Using the collected imaging data and analysis, it is possible to identify areas having similar characteristics. Together with knowing the location of individual organisms, information about how the crop changes and evolves over time may be monitored, and decisions taken for action where necessary. For example, when a leaf has reached a certain size, the crop can be moved to a harvest area. Conversely, if a crop is failing to grow as expected, remedial action may be taken or the environmental conditions may be changed to improve growth. It will be appreciated that such analysis may be improved with additional information about the crop in question, and information of the particular vertical farm.

The method may further comprise one or more steps of: instructing the data collection device to, at a predetermined position, capture an image, survey the depth and collect environmental data according to a schedule; and instructing the data collection device to calibrate the imaging means, depth sensor means and one or more environmental sensors.

As noted above, collecting data in a predetermined or specified location, allows crops to be monitored over a period of time. The schedule may be hourly, daily, or weekly for example, depending on the speed at which the crop typically changes or grows. In this way, the crop may be evaluated throughout its life cycle. Further, in order to ensure the data is accurate, the imaging means, depth sensor means and environment sensors may be calibrated.

To provide the map of the imaged area, the data processor may calculate a normalize difference vegetation index (NDVI) for each pixel of the image data to provide the map.

The NDVI data may be converted into a monochromatic image. It will be appreciated that since each sensor has its own characteristics and performances, in particular with respect to the position, width and shape of the spectral bands, a single formula like NDVI may yield different results when applied to the measurements acquired by different instruments. These effects may be alleviated to some extent by calibrating the senor means, as discussed above.

Establishing values for each pixel allows a high level of resolution on the crop. Analysis of the crop may be on the level of individual organisms. In the case of plants, analysis of the crop may be on the level of individual leaves. As noted above, individual leaves may be identified more easily identified by detecting light wave orientation using polarisation filter means and edge-based object recognition. As noted above, wide view data may be supplemented with more detailed data, for example, as collected by a probe.

A NDVI value may be used to better evaluate the collected data. Threshold values may be used to identify characteristics such as maturity. Other threshold values may be used to determine problems with crops, such as discolouration, for example caused disease.

By converting the NDVI values into a monochromatic data set or image, it may be possible to high-light the characteristics of interest.

In this way, individual areas, or organisms, or parts of organism may be identified having particular characteristics. The processor may identify areas with NDVI values corresponding to typical crop characteristics to evaluate crop health. By identifying problems on a small scale, it may be possible to take mitigating action before the problem spreads throughout the crop.

The method according may further perform one or more additional processes of: comparing the stored data with historic data; measuring one or more characteristic of a crop; counts or measures the number or area size of adjacent pixels having similar or the same value to evaluate size; forecasting crop results; using machine learning (ML) and or artificial intelligence (AI) to provide optimised conditions and cultivation processes within the vertical farm; using machine learning (ML) and or artificial intelligence (AI) to forecast, identify, suggest, update, improve, optimise and or facilitate growing condition experiments, formulas, and or recipes; and transmitting data to other data collection and monitoring systems used for other vertical farms.

Once data has been collected and analysed, the data set may be enhanced using historic data and other information about a specific type of crop. The historic data may be collected over time from the specific indoor farm, or the historic data may be collected from a number of other farms which may be growing crops under similar conditions, or other farms may be growing crops under different types of conditions. Data from the other farms may be collected and shared substantially simultaneously and used in real time, or data from other farms may be stored for later comparison.

Crop characteristics may comprise one or more of: total reflectance, relative spectrum reflectance, health state, colour, colour variation, uniformity, size, shape, density, surface texture, overall batch colour variation, overall batch uniformity, overall batch size, overall batch shape, overall batch density, overall batch distribution, overall batch surface texture, individual organism colour, individual organism colour variation, individual organism uniformity, individual organism size, individual organism shape, individual organism distribution, individual organism surface texture, plant size, plant shape, plant distribution, plant density, leaf colour, leaf colour uniformity, leaf size, leaf shape, leaf distribution, leaf density, leaf texture.

Machine learning (ML) and or artificial intelligence (AI) may be used to enhance monitoring and identification of crop characteristics.

The processor may use machine learning (ML) and or artificial intelligence (AI) to identify optimised environmental conditions at locations or positions within an area of operation for: batch yield, batch health, batch growth rate, sustaining batch readiness, controlling batch growth, total reflectance, relative spectrum reflectance, health state, colour, colour variation, uniformity, size, shape, density, surface texture, overall batch colour variation, overall batch uniformity, overall batch size, overall batch shape, overall batch density, overall batch distribution, overall batch surface texture, individual organism colour, individual organism colour variation, individual organism uniformity, individual organism size, individual organism shape, individual organism distribution, individual organism surface texture, plant size, plant shape, plant distribution, plant density, leaf colour, leaf colour uniformity, leaf size, leaf shape, leaf distribution, leaf density, leaf texture, crop or plant health, crop uniformity, plant size, plant shape, plant distribution, plant density, leaf size, leaf shape, leaf distribution, leaf density, leaf texture, crop quality, crop consistency.

Outputs provided by the processor may comprise: plant profiles, yield predictions, yield optimisation, growing ‘Flip Book’ of the crop over time, real-time alerts, disease detection, leaf identification, or remote inspection

Thus a data collection and monitoring system, a controlled environment farming system, devices and methods are provided for using data to improve farming systems.

Embodiments of the present invention will now be described, by way of example only, with reference to the accompanying drawings, in which like reference numerals are used for like features, and in which:

FIG. 1 is a representative drawing of a prior art growing system;

FIG. 2 is a representative drawing of another prior art growing system;

FIG. 3 compares the reflectance of light in visible blue, green, visible red and near-infrared for dead, stressed and healthy leaves;

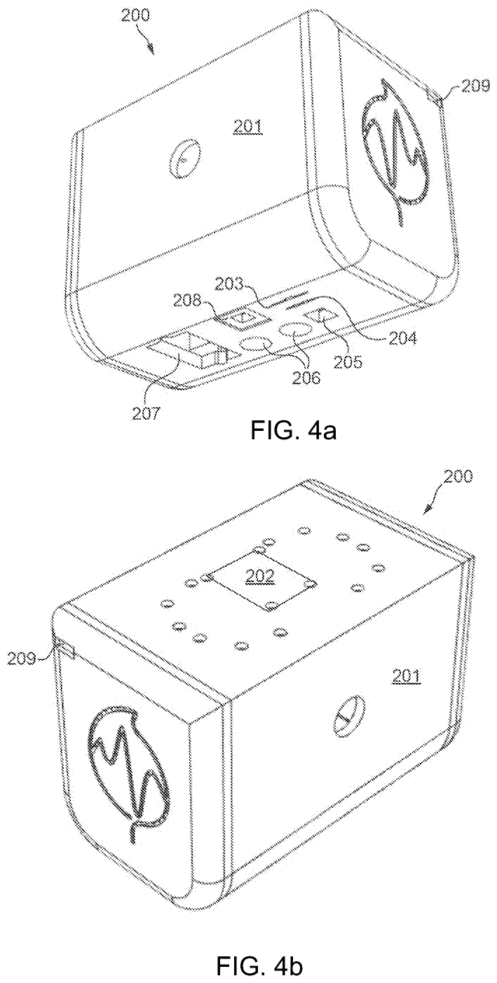

FIGS. 4a and 4b are perspective views of a data collection device;

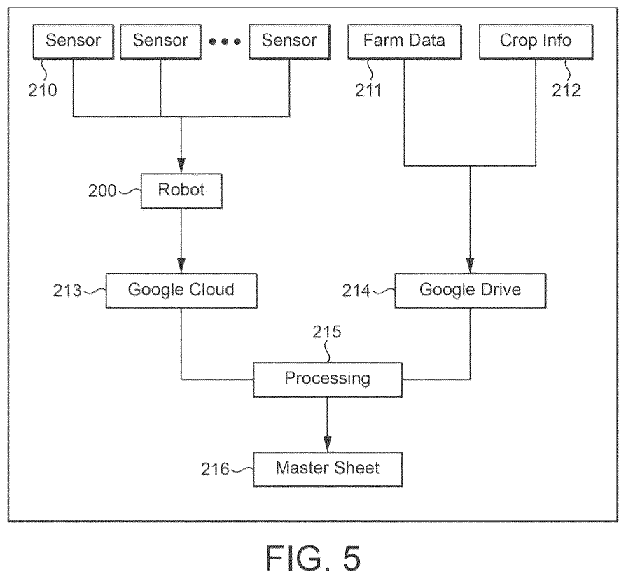

FIG. 5 is a representative flow diagram for data collection and processing;

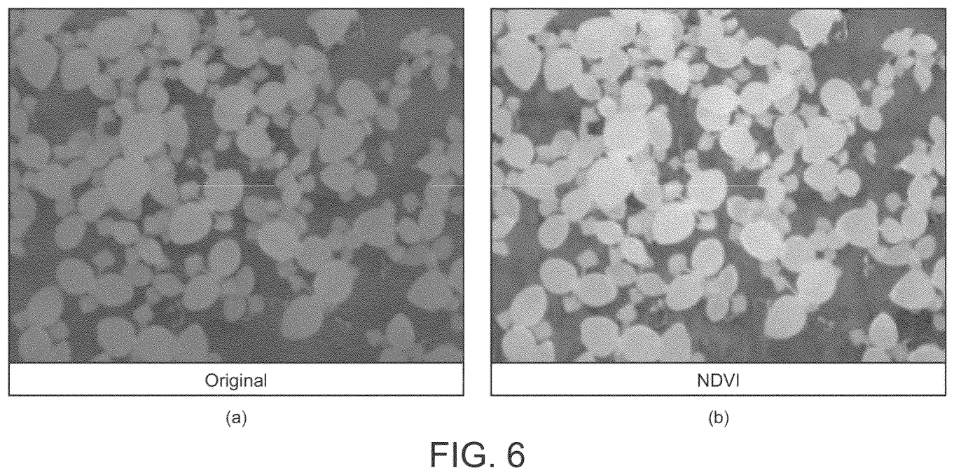

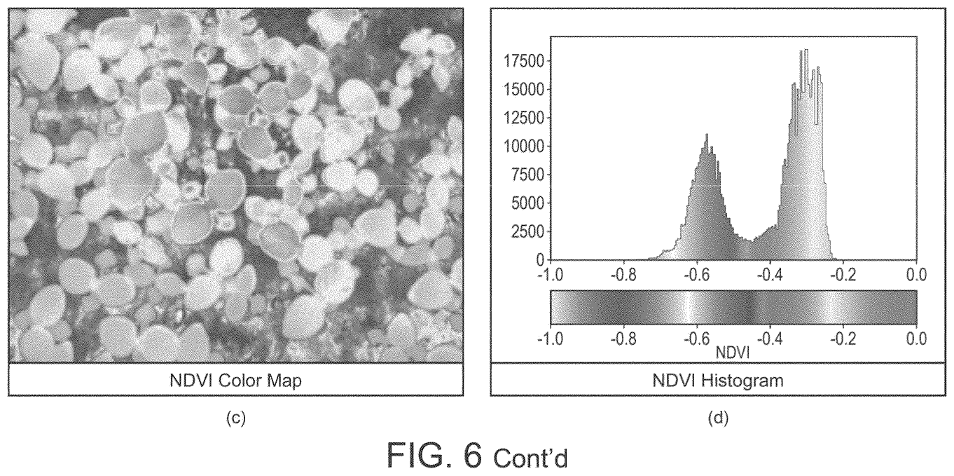

FIG. 6 shows illustrates processing steps in producing a map of an imaged area;

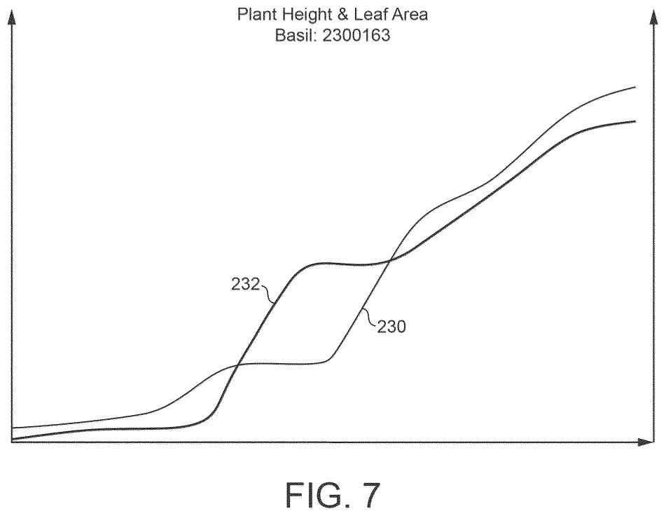

FIG. 7 is a graph of plant height and leaf area against time;

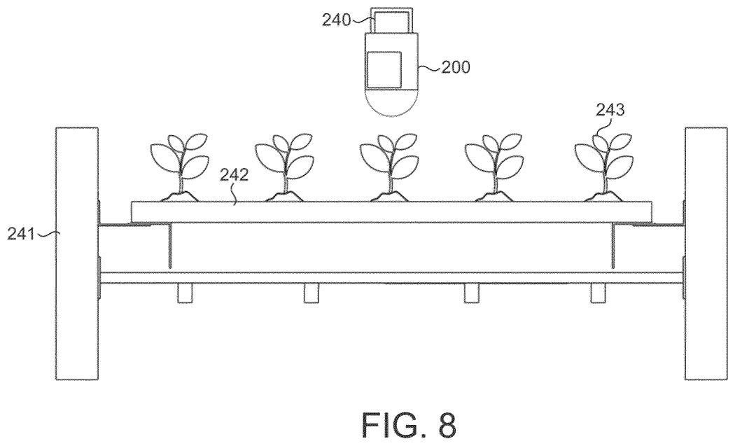

FIG. 8 illustrates a plane view of a data collection device in a farm having a one-dimensional rail system;

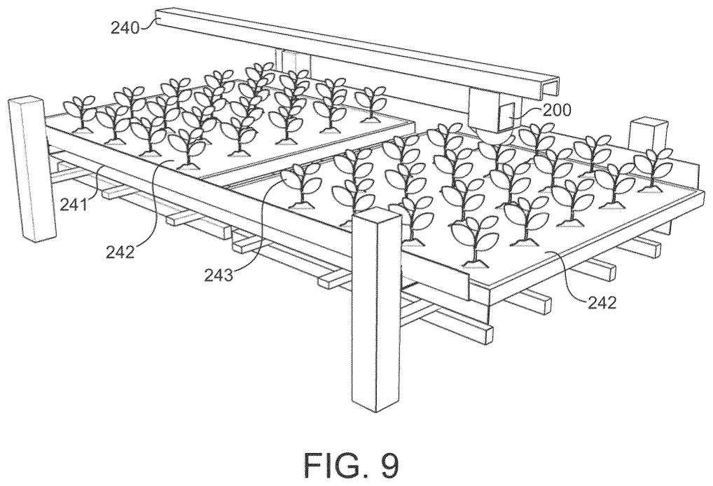

FIG. 9 illustrates a perspective view of a data collection device in a farm having a one-dimensional rail system;

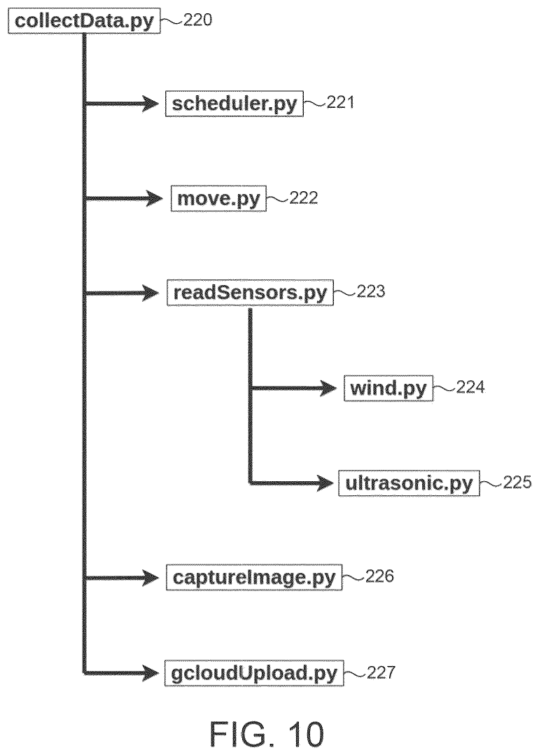

FIG. 10 is representative of control routine(s) used during data collection;

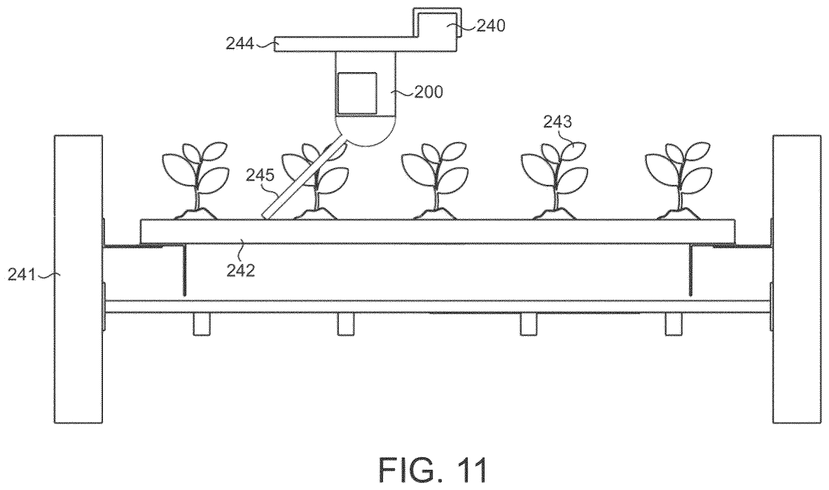

FIG. 11 illustrates a plane view of a data collection device having a probe in a vertical farm having a two-dimensional rail system;

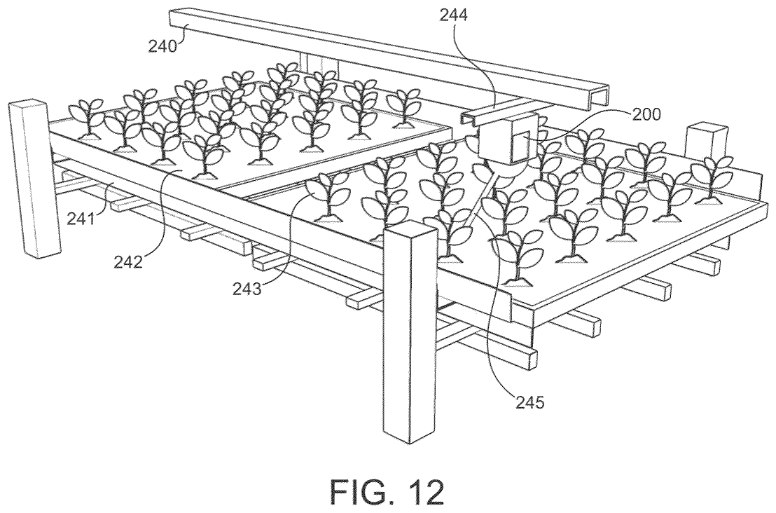

FIG. 12 illustrates a perspective view of a data collection device having a probe in a vertical farm having a two-dimensional rail system;

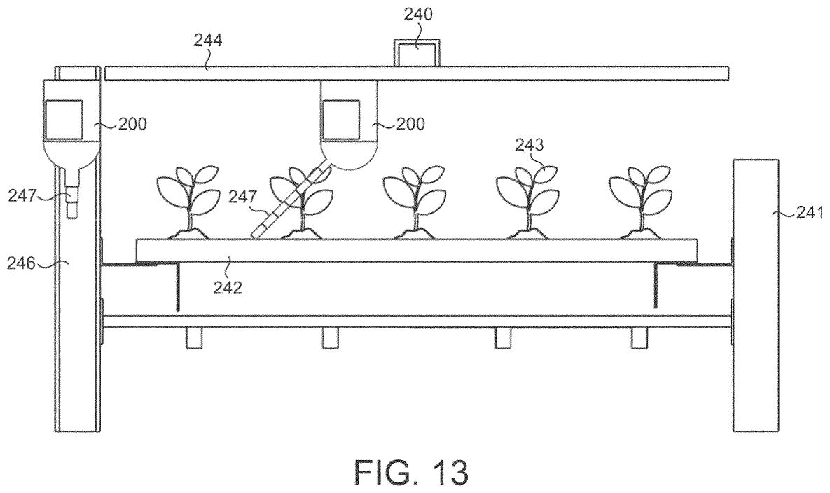

FIG. 13 illustrates a plane view of two data collection devices having retractable probes in a vertical farm having a three-dimensional rail system;

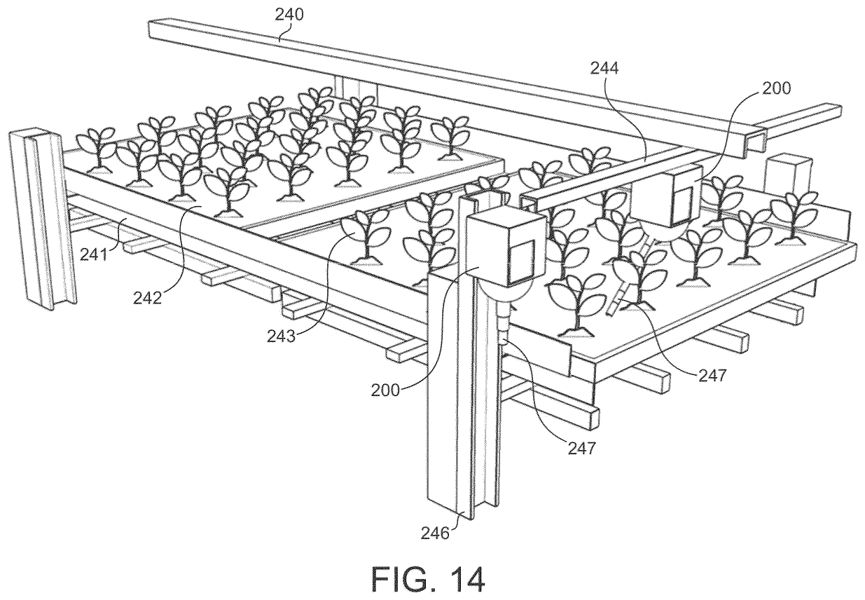

FIG. 14 illustrates a perspective view of two data collection device having probes in a vertical farm having a three-dimensional rail system;

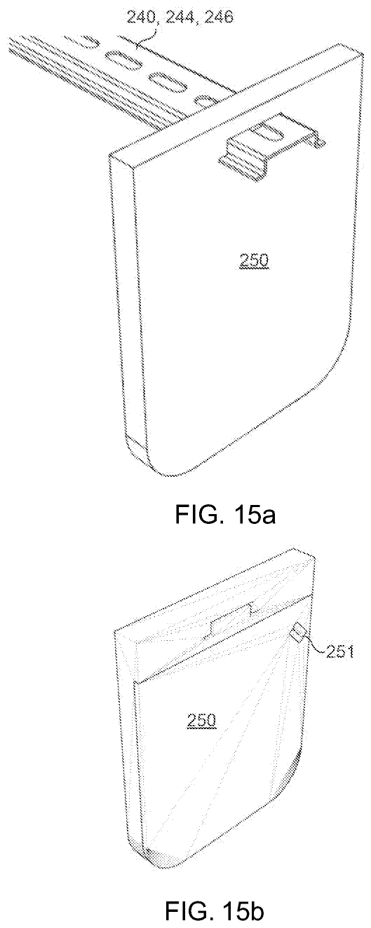

FIGS. 15a and 15b are perspective views of opposed faces of a wireless-charge unit, in FIG. 15a the unit is mounted on a rail;

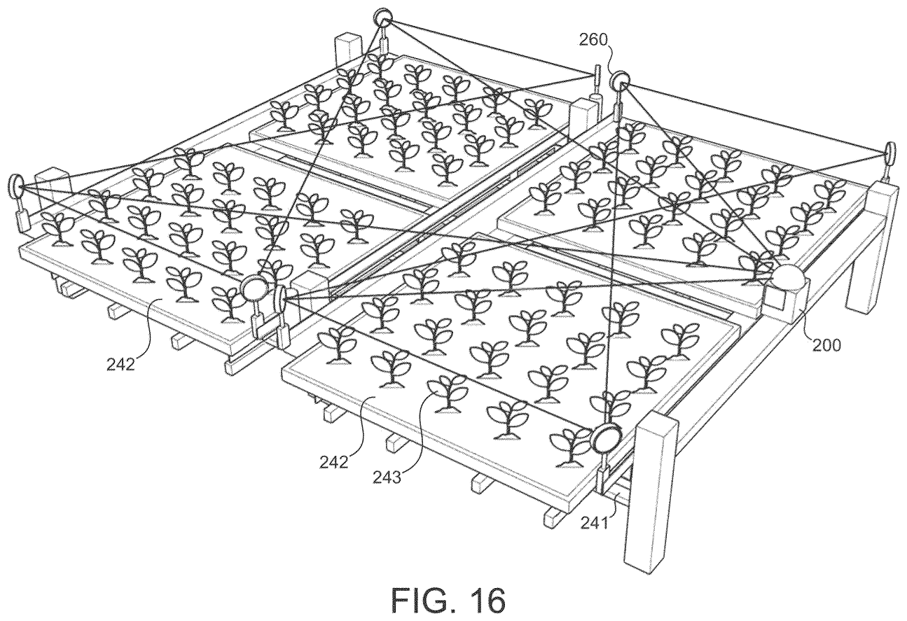

FIG. 16 illustrates a perspective view of a fixed data collection device in a vertical farm having a controllable mirror system;

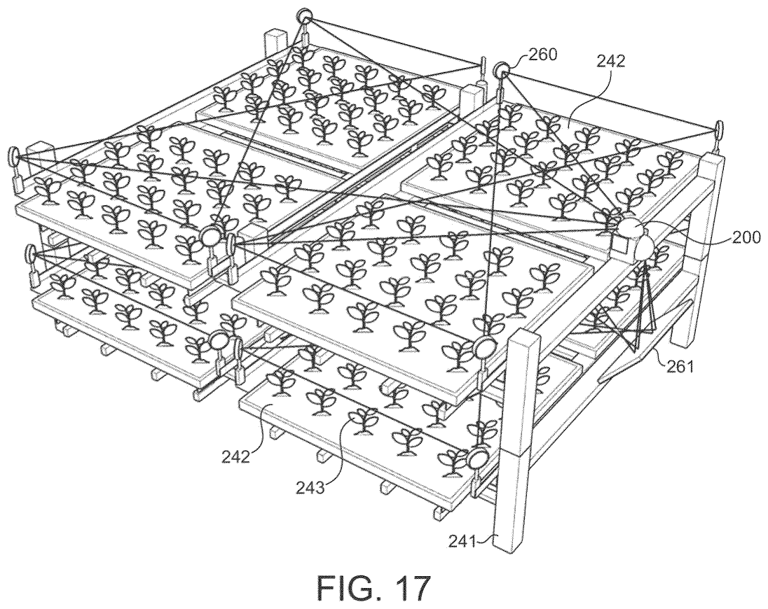

FIG. 17 illustrates a perspective view of a fixed data collection device in a vertical farm having a controllable mirror system operating over two racking levels; and

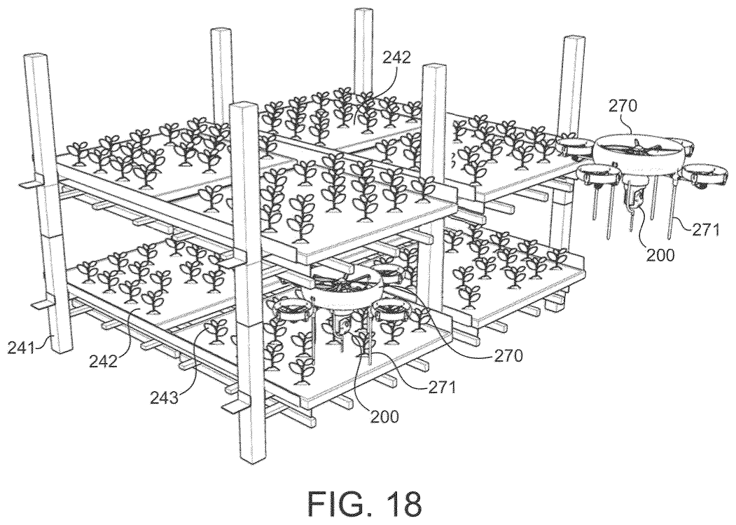

FIG. 18 illustrates a perspective view of two collection device mounted on a drones in a vertical farm operating over two racking levels.

### DETAILED DESCRIPTION OF DRAWINGS

The present invention may form part of a larger system. It will be appreciated that the system, methods and devices described herein are exemplary only, and other combinations and configurations of the apparatus and equipment described are anticipated by the inventors of the present disclosure without departing from the scope of the invention described here.

As noted above, FIGS. 1 and 2 are representative drawings of prior art indoor farming systems, and FIG. 3 illustrates reflectance of spectrum from leaves.

The devices, systems, and methods for improving data collection and analysis for an indoor farming system of the present invention are illustrated in the remaining drawings.

FIGS. 4a and 4b are front and back perspective views of a data collection device 200. As shown, the data collection device 200 has a substantially boxed shaped housing 201.

On the upper surface, the data collection device 200 has an RFID reader 202. The RFID reader 202 is used to detect location of the device 200 relative to growing trays and rack in the system.

On the lower surface or bottom of the data collection device 200 there are a number of openings in the housing 201 for the sensors to be exposed to the outside environment. The sensors are otherwise contained within the housing 201. The sensors comprise: a temperature sensor 203, a wind sensor 204, a barometer or pressure sensor 205, a depth, proximity or ultrasonic sensor 206, a combined CO₂/temperature/humidity sensor 207, a camera 208 which may be for visible light, near IR, IR or other spectrum frequencies, and the camera 208 may comprise polarization means. It will be appreciated that the data collection device 200 may comprise other sensors.

In addition, the housing 201 contains motors and wheels (not shown) for allowing movement of the data collection device 200, for example along a rail. In this way, the data collection device 200 is mobile.

It will be appreciated that although the data collection device housing 201 has been shown as substantially boxed shaped, the data collection device 200 have any suitable shape to contain the various sensors and other components.

It will be appreciated that the specific location each of the sensors with the data collection device 200 is not restricted, provided that the necessary openings are sufficient to monitor the environment proximal to the data collection device 200.

FIG. 5 is a representative flow diagram of data collection and processing. The data collection device or robot 200 collects data using the sensors 210 comprising sensors 203, 204, 205, 206, 207 and 208 as discussed above. The data collection device 200 transmits the collected data to a cloud storage system 213 for example Google Cloud, which is accessed and used by a processing module or processor 215. Typically the sensor data collected comprises: captured images, distance or depth survey to the crop canopy, and environmental conditions information for example CO₂ concentration, temperature, % humidity, pressure, and wind speed. In addition, the data collection device 200 identifies a particular tray and location thereof that is surveyed and records the location and the time of the data collection.

The processing module 215 also uses information provided as inputs, via a storage drive 214 such as Google Drive, which comprises Farm Data 212 and Crop Info 212. Farm Data 212 may comprise information in connection with the specific indoor farm, water pH used within the farm, water EC (electrical conductivity), and historic data such as yield from the farm. Crop Info 212 may comprise the plant species and typical growing requirements.

The processing module 215 uses the captured image data and the depth sensor data to provide a topographical map of the imaged area.

The camera 208 may be a single camera capturing a broad spectrum, including NIR, or the camera 208 may comprise more than one camera for separately capturing visible and NIR spectrums. Polarization filters may be used to capture images with specific light wave orientation. In the case of separate cameras, the images are taken at the same time and the information superimposed. FIG. 6 shows the steps in processing image data to produce the topographical map and assessed the area imaged. For each pixel of the image, the NDVI value between-1.0 and +1.0 is calculated, from imaged the Red and NIR reflectance, as shown in 6(a). The NVDI image array is converted into a monochromatic image as shown in 6(b). This is then converted into a false-colour image to emphasise areas with high NVDI values as shown in 6(c). Alternatively, the values for the area may be shown as a NDVI histogram (d). The lower and upper ends of the range of NDVI values may be adjusted according to the conditions, for example LED lights emit less near-IR than sunlight so the NDVI for a healthy leaf may be negative.

In this way, images of the crop may be used to create a map of the crop canopy based on reflectance in the visible and NIR spectrums. The image map may then be combined with the depth sensor survey of the area to create a topological map of the area. The processed data is stored against time, date, tray identify, tray position, time, CO₂, temperature, humidity, pressure, wind speed and height or depth in the master sheet 216.

Areas of pixels having the same NDVI are identified and used as an indication of leave size or area. Alternatively total leaf area may be calculated from the proportional of pixels above a threshold value.

It will be appreciated that the resolution of the camera images is such that individual plants and leaves may be identified. It will be appreciated that the NVDI may indicate, biomass, chlorophyll concentration in leaves, plan productivity and cover. In some cases, it will be possible to identify variation in colour over single leaves. Together with known information about the plant type, variation in colour of a leave may be an indication of plant health or disease.

When data is collected from the same position over a period of time during the crop's life cycle, it is possible to track plant growth in terms of leaf area and height of plant.

FIG. 7 is a graph of plant height and leaf area (y-axis) against time (x-axis), where the plot 230 shows the height of a selected plant, and plot 232 shows the leaf area of that plant. It will be appreciated, that as a seedling, initially the plant has relatively few and small leaves and puts effort into increasing in height. Once the plant reaches a threshold height effort is switched from gaining height to increasing the number and area of leaves for absorbing more energy. After this, the height and leaf area increase together until the rate of increase begins to flatten as the plant reaches maturity.

The calculated crop characterises such as, the crop height, leaf area and normalised difference vegetation index NDVI or plant profiles are stored in a database 216 or Master Sheet as data processor outputs which may be called on by a farming system controller. Data processor outputs may also comprise: plant profiles; yield predictor; yield optimisation; growing flip book; real time alerts; disease detection leaf identification; and remote inspection

FIGS. 8 and 9 illustrates a data collection device 200 in use in a vertical farm. Within the growing room of a vertical farm, a number of growing trays 242 are arranged on a rack 241. The growing trays 242 contain a crop of plants 243. Above the area where growing trays 242 are placed, a rail 240 extends along the length of the rack 241. The data collection device 200 is supported by the rail 240. Further the data collection device 200 is arranged to be moveable along the rail 240. In this way, the data collection device 200 may be positioned above either a first tray 242 or a second tray 242 to collect data on with a wide view about the crop in each tray 242. However, it will be appreciated, that the imaging means captures images with a large number of pixels such that individual features can be identified from the wide view.

FIGS. 11-14 and 16-18 illustrate various alternative arrangements for positioning the data collection device 200 within the vertical farm for viewing a crop, and collecting data from different growing areas on the rack 241 of the vertical farm.

FIGS. 11 and 12 illustrate a data collection device having a probe, and where the vertical farm has a two-dimensional rail system. As illustrated, the data collection device further comprises a probe 245 affixed to the bottom side of the data collection device 200. The probe 245 extends towards the crop 243. Additional data collectors, such as additional imaging means, depth or distance sensor means, environmental sensors, and or location sensor means, are located towards the distal end of the probe. In this way, data about the crop or individual plants 243 may be collected from a position closer to individual plants 243 and from different angels. It will be appreciated that the wide view from above may identify areas of interest, and the probe 245 may be used to get closer to the areas of interest to capture yet more detail for analysis. The probe 245 may be long enough to reach the growth tray 242.

In this way, data may be collected from amongst the plants 243 or even from the substrate which supports the plants 243. The probe may capture close up images to identify crop diseases for example. It will be appreciated that the probe may be rotated so that it can reach more areas of the crop, and from different angles as required for identifying and locating particular problems.

In addition to the probe 245, the arrangement in FIGS. 11 and 12 illustrate a second rail 244, arranged perpendicularly to the first rail 240. With this arrangement, the data collection device can move in two-dimensions, in the x-direction along the first rail 240 and in the y-direction along the second rail 244. The two-dimensional rail system extends the area which is reachable by the data collection device 200. In this way, the data collection device 200 may have several vantage points above a single growth tray 242 for collecting data, thereby increasing the amount of data and detail that may be collected. It will be appreciated, that the data collection device 200 may move along each of the first and second rails 240, 244, or the data collection device 200 may be fixed to the second rail 244 and the second rail may move relative to the first rail 240.

Another arrangement is illustrated in FIGS. 13 and 14 which show two data collection devices having retractable probes 247, operating on a three-dimensional rail system. The retractable probe 247 is similar to probe 245. However, the retractable probe 247 may be retracted when not in use, and extended as required.

The three-dimensional rail system comprises a third or vertical rail 246 arranged vertically in the z-direction substantially parallel with an upright of the rack 241. As shown, a second data collection device 200 is operating on a vertical rail 246, while the first data collection device 200 operates on the x- and y-direction rails 240, 244. Vertical rails 246 may be arranged adjacent to each of the rack uprights, or the number of vertical rails 246 may be limited. As shown, the vertical rail 246 is not connected to the first and second rails 240, 244 and therefore the data collection devices 200 cannot move between the horizontal rails 240, 244 and the vertical rail 246. However, a three-dimensional rail system where a data collection device may move between each of the rails is anticipated.

It will be appreciated that the data collection device 200 may be powered by an on-board battery or super capacitor. FIG. 15a illustrates a wireless-charge unit 250 that is mounted on a rail 240, 244, 246. It will be appreciated that wireless-charge units 250 may be mounted on any rail, and typically will be mounted at the end of a rail so as not to interfere with movement of the data collection device 200 along the rails 240, 244, 246. FIG. 15b shows the opposed side of the wireless-charge unit 250. In use the wireless-charge unit 250 is mounted such that the opposed side faces the operational length of the rails 240, 244, 246. When a data collection device 200 is low on power, the device 200 moves along the rails 240, 244, 246 to butt against or to be proximal to the wireless-charge unit 250. Charging may begin when the data collection device 200 triggers the switch 251 on the face of the wireless-charge unit 250.

FIG. 16 illustrates an arrangement where the data collection device 200 is in a fixed position. As shown, four growth trays 242 are arranged in a 2×2 configuration, and the data collection device is mounted on the rack 241 roughly at the mid-point of one edge of the trays 242. Mirrors 260 are mounted at the corners and tray edges. The mirrors 260 are arranged to interact with the data collection device 200 by directing images to the data collection device from substantially all of the area of the growth trays 242. Typically images from different areas of the trays may be collected, while environment data may be collected as a single value for all of the trays. In some arrangements, the angular direction of the mirrors may be adjustable to direct the image signal from a slightly different area to the data collection device 200. It will be appreciated that in order to reach all areas, it may be necessary for the image to be re-directed using more than one mirror 260. Accordingly it may be necessary for the mirrors 260 to be coordinated to work together. As a result, typically the mirrors 260 may be centrally controllable by a farm system controller.

FIG. 17 illustrates an extension of the arrangement shown in FIG. 16, where the rack 241 comprises two levels each having mirror arrangement 260. A single data collection device 200 serves both levels using another mirror 261 angled to direct images between the levels.

FIG. 18 illustrates another multi-level rack arrangement, where the levels are suitably spaced to allow a drone 270 to pass between the levels. A data collection device 200 is mounted on the drone 270. In this way, any tray 242 may be reached by the data collection device 200. The drone 270 may be provided with supports 271 to allow the drone to set down on a tray 242 with the data collection device 200 a fixed distance above the tray surface. In a set down position, the data collection device 200 may be instructed to collect images, depth and other information.

FIG. 10 is representative of a control collectData program 220 used to control the data collection device 200 during data collection. The collectData program 220 is the main control of the data collection device 200. The collectData program 220 carries out the task of moving the data collection device 200 to a predetermined position at a scheduled time, taking senor readings and capturing images.

Initially a scheduler module 221 will schedule the program to start collecting data. A move module 222 commands the data collection device 200 to move to a specific position within the indoor farm. The data collection device 200 moves along a rail until an RFID tag is detected. Optionally, the data collection device 200 may stop at additional positions above a particular tray. For example, the specific position may be a specified distance from the edge of the tray where the RFID tag is located. When the data collection device 200 arrives at the position, the readSenors module 223 collects data using the wind module 224 and ultrasonic module 225. Typically, a number of sensor readings may be taken at the position to provide an average result. At a similar time, the capturelmage module 226 is used to collect images at the position. Collected data may be stored locally in a file. Once the data has been collected, the file is uploaded to the Cloud by the gcloudUpload module 227. Typically once the file has been uploaded the file will be deleted locally to save data storage space on the data collection device 200. Data may be uploaded daily, or more or less frequently according to requirements.

It will be appreciated, that the CollectData program 220 may comprise other modules for collecting data from the sensors available within the data collection device 200. It will be appreciated that the collect data program 220 is repeated according to the scheduler module 221 which may be controlled by a central control facility. The collectData program 220 may further comprise a return-to-base function to move the data collection device 200 to a charge station when the power supply drops below a threshold charge.

In this way, the information about the crop in the indoor farm may be gathered over time and used to analyse the crop.

It will be appreciated that the data collection and analysis provides the ability to accurately assess the healthiness of the plant at different stages of growth of the plant. By determining the healthiness of the plant during its growth cycle, it is possible to tailor the environment to suit the plant during its growth. It will be appreciated that different environmental conditions and nutritional provisions may be required at different times during the growth cycle, and the ability to adjust these according to real time data collection and analysis may provide a healthier plant, and importantly a more economically viable crop. Environmental condition and nutritional requirement parameters can be measured and adjusted at the growth tray level and help to improve consistence across the farm and between crop cycles.

Using historical data, it is possible to create expected growth profiles for different types of plants. Correlated with the data gathered of the specific environment around the plants, growing conditions for the crop may be optimised for the health of the plants. It will be appreciated that this data also enables predictions and optimisation of yield of the crop. In this way, crop “recipes” which mix the correct amount of all the variables may be created for indoor farms.

It will be appreciated that the data collection device increases automation within the indoor farm, and enables remote inspection of the vertical farm, and can be used for early detection of problems with crop, such as disease. Furthermore, the data collection device minimises the need for operators to enter the growing room, which may be a high-care environment. In turn, reduced operator activity in the growing room, reduces the possibility of introducing pests and diseases into the growing room.

It will be appreciated that, the devices, systems, and methods for improving data collection and analysis for a farming system and growing facility described herein provides information that may be used by crop growers. Accordingly, the devices, systems, and methods may provide improvements in efficiency, quality and use of space within an indoor farm, for example.

It will be appreciated that, advantageously, the arrangement requires minimal interaction or connectivity between components. Accordingly, the growing facility may be relatively cheap, and straight-forward to run without worker intervention, and thus reduces the risk of microbiological contamination. The arrangement may be particularly advantageous in high-care environments, where growing rooms are kept to a high standard of cleanliness.

It will be appreciated that the arrangement could be retrofitted to existing indoor farming facilities.

It will be appreciated that the arrangement could be integrated with irrigation, and environment subsystems within a facility and automatically adjust schedules according to feedback provided by collected data. Advantageously, it will be appreciated that customisation of the subsystems allows the facility to meet short-term fluctuation in demand.

Advantageously, it will be appreciated that they are no direct couplings required between the growing and the data collection device.

The farming system described above with reference to the figures allows for data monitoring and therefore control of the growing environment. Accordingly crop yields and growing times may be improved, contamination may be minimised, and product shelf life may be optimised.

Whilst endeavouring in the foregoing specification to draw attention to those features of the invention believed to be of particular importance, it should be understood that the applicant claims protection in respect of any patentable feature or combination of features referred to herein, and/or shown in the drawings, whether or not particular emphasis has been placed thereon.

It will be appreciated that a farming system, method and devices can be designed for a particular application using various combinations of devices and arrangements described above. It will be appreciated that the features described hereinabove may all be used together in a single system. In other embodiments of the invention, some of the features may be omitted. The features may be used in any compatible arrangement. Many variations and modifications not explicitly described above are possible without departing from the scope of the invention as defined in the appended claims.

In this document, the language “movement relative to a gap” is intended to include movement within the gap, e.g. sliding along the gap, as well as movement into or out of a gap.

In this document, the language “movement in the n-direction” (and related wording), where n is one of x, y and z, is intended to mean movement substantially along or parallel to the n-axis, in either direction (i.e. towards the positive end of the n-axis or towards the negative end of the n-axis).

In this document, the word “connect” and its derivatives are intended to include the possibilities of direct and indirection connection. For example, “x is connected to y” is intended to include the possibility that x is directly connected to y, with no intervening components, and the possibility that x is indirectly connected to y, with one or more intervening components. Where a direct connection is intended, the words “directly connected”, “direct connection” or similar will be used. Similarly, the word “support” and its derivatives are intended to include the possibilities of direct and indirect contact. For example, “x supports y” is intended to include the possibility that x directly supports and directly contacts y, with no intervening components, and the possibility that x indirectly supports y, with one or more intervening components contacting x and/or y.

In this document, the word “comprise” and its derivatives are intended to have an inclusive rather than an exclusive meaning. For example, “x comprises y” is intended to include the possibilities that x includes one and only one y, multiple y's, or one or more y's and one or more other elements. Where an exclusive meaning is intended, the language “x is composed of y” will be used, meaning that x includes only y and nothing else.

1. A data collection and monitoring system for assessing a crop of living organisms in a controlled environment farming system, the collection and monitoring system comprising:
a data collection device having an imaging means, and one or more environment sensors; and
a data processor means configured for receiving collected data from the data collection device, wherein based on collected data the data processor means is configured to provide mapped information of an imaged area combined with collected environmental data as an output.

2. A data collection and monitoring system according to claim 1, wherein imaging means comprises:
at least one or more of a polarising means, a wide angle lens, a fish-eye lens, and/or a LIDAR device for capturing data in three-dimensions.

3. A data collection and monitoring system according to claim 1, wherein the imaging means captures wide spectrum images comprising:
at least one or more of a visible light and/or non-visible light.

4. A data collection and monitoring system according to claim 1, wherein the one or more environmental sensors comprise:
at least one or more of a wind sensor means; CO₂ sensor means; temperature sensor means; humidity sensor means; pH sensor means; conductivity sensor means; a combined CO₂/Temperature/Humidity sensor; a combined temp/pressure/humidity sensor; and/or pressure sensor means.

5. A data collection and monitoring system according to claim 1, wherein the data collection device comprises:
a probe having at least one or more of imaging means, a depth sensor means, one or more environmental sensors, and/or a location sensor means located towards a distal end of the probe.

6. A data collection and monitoring system according to claim 1, wherein the data collection device comprises:
movement means configured for moving within an area or volume of operation.

7. A data collection and monitoring system according to claim 1, wherein the data collection device comprises:
a depth sensor means, and the data processor is configured to provide topographical mapped information of an imaged area combined with collected environmental data as an output.

8. A data collection and monitoring system according to claim 7, wherein the depth sensor means comprises:
an acoustic sensor.

9. A data collection and monitoring system according to claim 7, wherein the imaging means and depth sensor are combined to capture data in three-dimensions.

10. A data collection and monitoring system according to claim 1, wherein the data collection device comprises:
a location means.

11. A data collection and monitoring system according to claim 1, in combination with a controlled environment farming system, the combination comprising:
the data collection device; and
the data processor means configured for use in the data collection and monitoring system;
wherein the data collection device is positioned to survey an area for receiving a crop and the data processor is configured to provide an output including mapped information and environmental data.

12. A combination having controlled environment farming system according to claim 11, wherein the data processor is configured to provide an output comprising:
topographical mapped information and environmental data.

13. A combination having a controlled environment farming system according to claim 11, wherein the processor is remotely located relative to the data collection device.

14. A combination having a controlled environment farming system according to claim 11, wherein the processor is operationally in communication with the data collection device and a controller is arranged to respond to the data processor output.

15. A combination having a controlled environment farming system according to claim 11, wherein the data collection device is configured to self-calibrate calibrate at least one or more of the: imaging means, depth sensor means, environmental sensor(s) and/or location means against one or more known references located within a vertical farm.

16. A combination having a controlled environment farming system according to claim 15, wherein the data collection device is configured to interface with ID tags located within the vertical farm to determine the location of the data collection device.

17. A combination having a controlled environment farming system according to claim 11, comprising:
a framework, racks and/or trays which include identity tags to sense location within an area of operation.

18. A combination having a controlled environment farming system according to claim 11, comprising:
a rail system extended in at least a first direction wherein the data collection device is movably mounted on the rail system for surveying an area.

19. A combination having a controlled environment farming system according to claim 15, wherein the data collection device or sensors are fixedly mounted within the vertical farm.

20. A combination having a controlled environment farming system according to claim 11, comprising:
one or more controllable mirrors mounted within the vertical farm, wherein the controllable mirrors are pivotable to direct line-of-sight of the data collection device a larger area of the controlled environment farming system.

21. A combination having a controlled environment farming system according to claim 11, wherein the data collection device is mounted on a drone.

22. A method of collecting and monitoring data in a for assessing a crop of living organisms in a controlled environment farming system, the collection and monitoring system comprising:
a data collection device having an imaging means, and one or more environment sensors; and
a data processor means configured for receiving collected data from the data collection device, wherein based on collected data the data processor means is configured to provide mapped information of an imaged area combined with collected environmental data as an output, the method comprising:
instructing the data collection device to collect image data and/or depth survey data, and environment data;
transmitting the collected data to the data processor,
wherein the data processor is configured to use the image data to provide a map of the imaged area, and
wherein the data processor stores the map with the collected environmental data.

23. A method according to claim 22, comprising:
collecting the image capture, depth survey and or the environmental data at a same time.

24. A method according to claim 22, comprising:
recording the location of data collection device when data is collected and stored with the map and the collected environmental data.

25. A method according to claim 22, comprising:
transmitting outputs of the data processor to a controller.

26. A method according to claim 22, wherein the data processor is configured to receive inputs including crop data; or
wherein the data processor receives inputs including vertical farm data.

27. A method according to claim 22, comprising one or more of:
instructing the data collection device to, at a predetermined position, capture an image, survey the depth and or collect environmental data according to a schedule; and
instructing the data collection device to calibrate the imaging means, depth sensor means and/or one or more environmental sensors.

28. A method according to claim 22, wherein to provide the map of the imaged area, the data processor is configured to calculate a normalize difference vegetation index (NDVI) for each pixel of the image data to provide the map.

29. A method according to claim 22, wherein the data processor is configured to perform one or more additional processes of:
comparing stored data with historic data;
measuring one or more characteristic of a crop;
counting or measuring a number or area size of adjacent pixels having similar or a same value to evaluate size;
forecasting crop results;
using machine learning (ML) and or artificial intelligence (AI) to provide optimised conditions and cultivation processes within the vertical farm;
using machine learning (ML) and/or artificial intelligence (AI) to forecast, identify, suggest, update, improve, optimise and or facilitate growing condition experiments, formulas, and or recipes; and
transmitting data to other data collection and monitoring systems used for other vertical farms.
